(19) United States
(12) Patent Application Publication
(10) Pub. No.: US 2004/0020710 A1
(43) Pub. Date: Feb. 5, 2004

(54) Stented ear mold and method

(76) Inventor: Monte Martinez, Castro Valley, CA (US)

Correspondence Address: Aldo J. Test, DORSEY & WHITNEY LLP, Suite 3400, 4 Embarcadero Center, San Francisco, CA, 94111, US

(21) Appl. No.: 10/414,858

(22) Filed: Apr. 15, 2003

Related U.S. Application Data
(60) Provisional application No. 60/373,699, filed on Apr. 17, 2002.

(51) Int. Cl. H04R 25/02; A61B 7/02

(52) U.S. Cl. 181/130; 181/135

(57) ABSTRACT

An ear mold with a tubular passage adopted to fit into the ear canal is described. A stent adapted to be inserted in the ear molds tubular passage for increasing the outer diameter of a loose fitting ear mold is provided. The stented ear mold prevents acoustic feedback.

### RELATED APPLICATIONS

This application claims priority to provisional Application Serial No. 60/373,699 filed Apr. 17, 2002.

### BRIEF DESCRIPTION OF THE INVENTION

This invention relates to a stented ear mold and ear mold fitting system providing a fitted ear mold and method for adjusting the size of an ear mold to fit tightly in the ear of a user and to prevent acoustic feedback.

### BACKGROUND OF THE INVENTION

Hearing aid devices are well known. Generally, they comprise a microphone, an amplifier, a control for adjusting the volume, a transducer and a battery. The microphone receives sound and converts it to an electrical signal which is amplified and applied to the transducer. The transducer transmits amplified sound waves into the ear canal towards the ear drum. An output or speaker tube is connected to the ear mold fitted in the ear canal to transmit the sound through a passage in the ear mold to the ear drum. A difficulty encountered with conventional hearing devices is related to obtaining a proper fit of the ear mold in the ear canal. If the ear mold is not properly fitted acoustic feedback results. That is, sound waves travel back along the ear canal between the ear mold and ear canal to the microphone where they are re-amplified. This builds up and the amplified sound may result in the whistling noise in the user's ear. This is annoying and interferes with the ability to hear. Impressions or casts are made of user's ear canals, which serve as the mold for fabrication devices having exterior surfaces which custom fit into the ear canal. In some instances the fabrication of the ear mold may result in improperly fitting. Even with properly fitted ear molds movement of the jaw during conversation, aging of the ear mold, etc. may change and alter the fit and provide the space for feedback. There is a need for a method of re-establishing the ear mold dimensions so that they properly fit into the ear canal.

### OBJECTS OF THE INVENTION

It is an object of the present invention to provide a stented ear mold and method for improving ear mold fit.

It is another object of the invention to provide a method in which a stent is inserted into the ear mold to adjust the diameter of the ear mold so that it fits tightly and comfortably in the ear canal.

The foregoing and other objects of the invention are achieved by an ear mold which includes an inserted stent that increases the diameter of an ear mold so that it fits tightly and comfortably in the ear canal of the user.

The invention also provides a method of converting loose fitting ear molds to tightly fitted ear mold by the insertion of stents which increase the outer diameter of the ear mold to assure a good fit in the ear canal.

### BRIEF DESCRIPTION OF THE DRAWINGS

The foregoing and other objects of the invention will be more clearly understood from the accompanying drawings and description of the invention:

FIG. 1 shows an ear mold inserted in the ear canal;

FIG. 2 shows a loose fitting ear mold installed in the ear canal;

FIG. 3 shows an ear mold with an inserted stent in accordance with the invention inserted in the ear canal;

FIG. 4 is an enlarged view of a typical stent;

FIG. 5 shows a stent insertion tool;

FIG. 6 shows a stent removal tool;

FIGS. 7a-7d illustrate the steps followed for inserting a stent into an ear mold; and

FIGS. 8a-8c show the step followed for removing or replacing a stent.

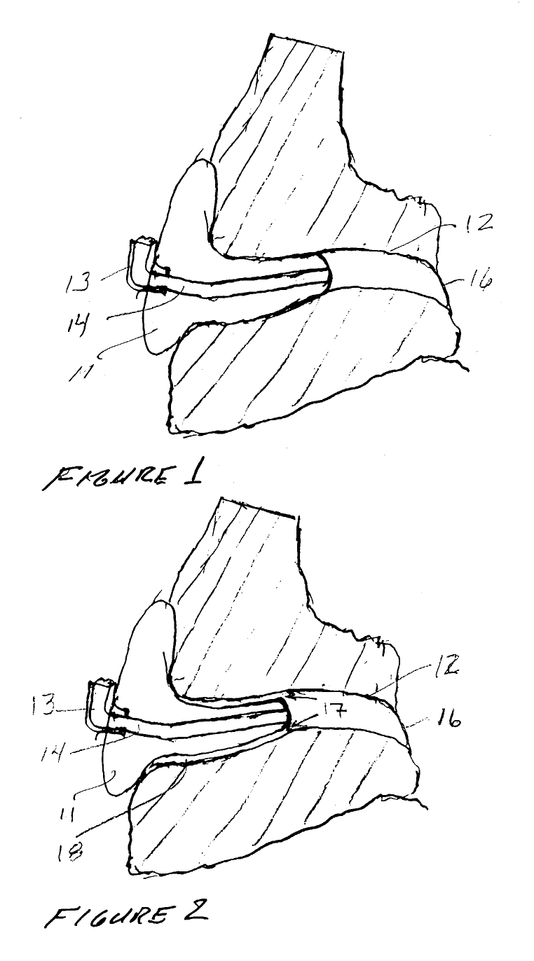

### DESCRIPTION OF PREFERRED EMBODIMENT

The ear mold 11 is molded to fit comfortably in the ear canal 12. A hearing aid connected to the fitting 13 introduces sound into the tubular passage 14 formed in the ear mold. The sound travels along the tubular passage to the ear drum 16. The ear mold is securely fitted into the ear canal to prevent sound from leaking through the canal back to the microphone which would cause undesirable feedback.

However, if the ear mold does not fit tightly into the ear canal the sound will leak back between the ear mold 11 and ear canal 12 as shown by the arrows 17, FIG. 2. This gap or feedback passage 18 may be caused by an improperly fabricated ear mold or it may arise from wear and tear or from aging. In any event, rather than discarding the ear mold and replacing it with another the present invention provides a stent and tools for stent insertion and removal. The stent changes the diameter of the ear mold whereby it can again properly fit into the user's ear canal and prevent acoustic feedback.

Referring particularly to FIG. 3, a stent 21 has been inserted into the ear mold tubular passage 14 and has caused the loose fitting ear mold of FIG. 2 to be enlarged from the size shown in FIG. 2 to the size shown in FIG. 3. With this change in size, the ear mold is again properly in the ear canal and prevent feedback.

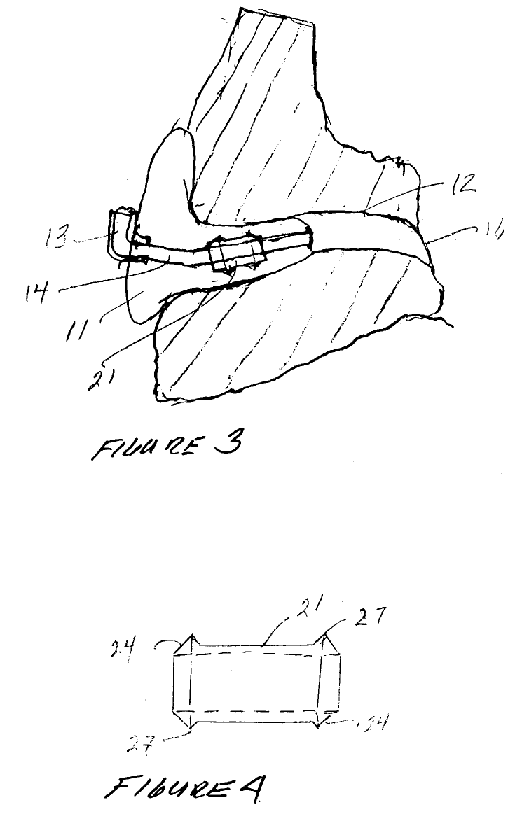

FIG. 4 shows a suitable ear mold stent. The stent has tapered ends 24 for ease and insertion and removal of the stent from the tubular passage. The ear mold includes barbs 27 which inhibit movement of the stent after it has been inserted. The internal diameter of the stent is substantially the same as the diameter of the tubular passage to provide a smooth passage from the hearing, and to the ear drum. Of course, it is to be realized that stents of different sizes are provided to permit tailoring of the ear mold diameter to properly fit into the ear canal. This accommodates loose fits of different degrees.

The stent may be used on brand new ear molds when feedback occurs due to loose fit or on old ear molds which have begun to produce feedback due to material shrinkage, etc. To avoid damage to the mold, the walls of the canal around the sound passage should be inspected. If the walls feel thin there may be a break through the wall when inserting a stent or possibly the creation of a protrusion which might cause irritation. This is especially true in molds that have an extremely small outer dimension.

FIG. 5 shows a ear mold insertion tool 31 which includes a handle 32, and stem 33 that terminates in a region of reduced diameter 34 to receive and hold a stent. FIG. 6 shows an ear mold removal tool 36 which includes a handle 37, a stem 38, and a slot 39, which is formed at the end of the tool to accommodate a stent during removal.

Ideally the stent should be placed so that the deepest part of the stent sits even or slightly above where the bowl and canal meet. Understandably, this is not always possible due to the wall thickness or angle of the tubular passage. The stent will work when placed anywhere in the tubular passage of the mold.

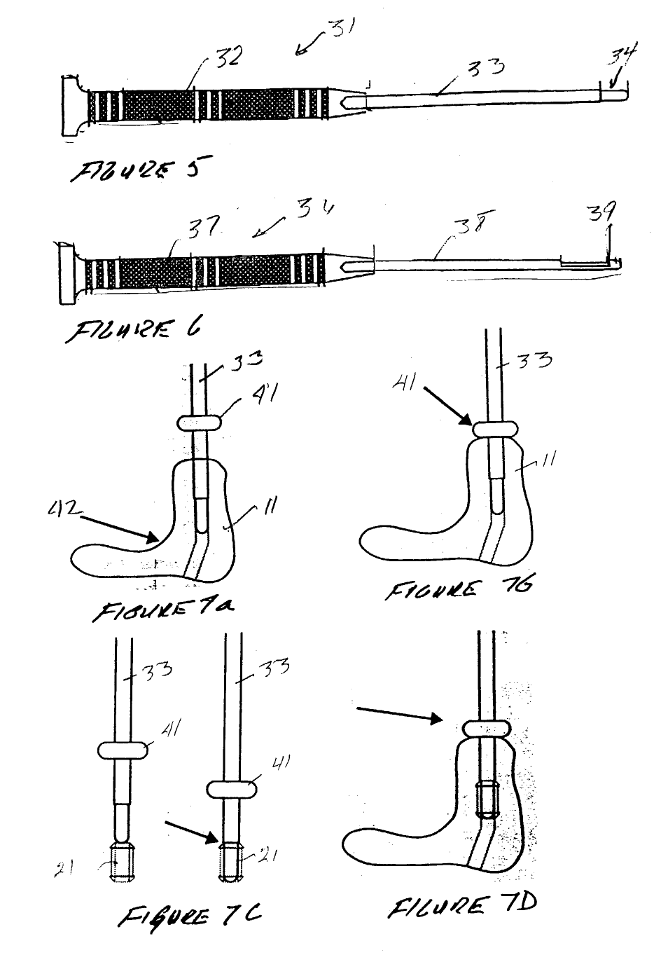

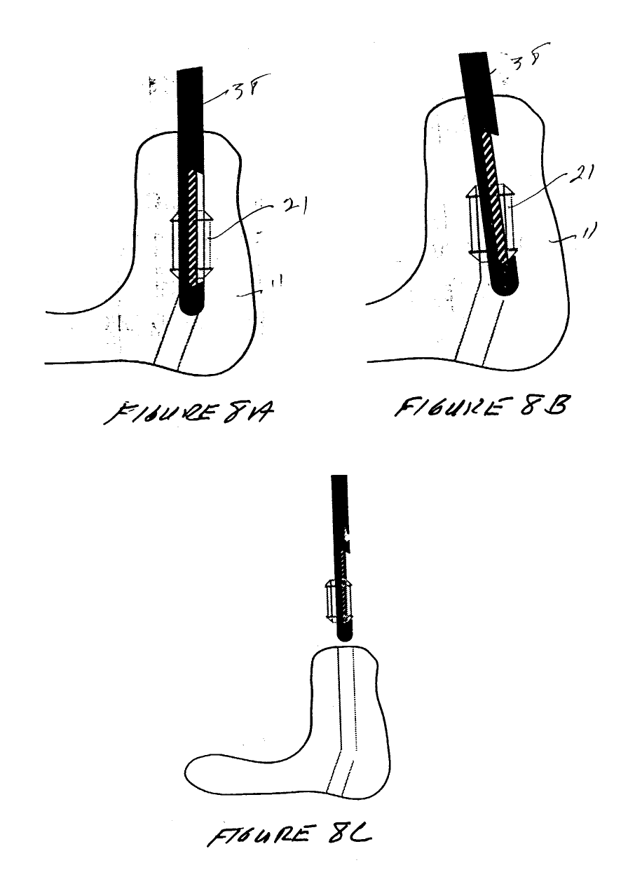

The steps in proper insertion of a stent into the ear mold are illustrated in FIGS. 7a-7d. Using the tool shown, FIG. 5, and o-ring 41 is threaded onto the stem as shown in FIG. 7a. The tool is then placed alongside the ear mold with the tip at the point where the canal opens into the bowl of the ear mold designated generally by the numeral 42. The o-ring is then slid down so that it is even with the top of the ear mold, FIG. 7b. The stent is placed on the end of the insertion tool so that it is completely onto the tool, FIG. 7c, and by slowly and gently working the tool the stent is inserted into the tubular passage making sure that it is not forced through the wall of the ear mold as it is inserted. The insertion is stopped when the o-ring reaches the top of the ear mold, FIG. 7d. The tool is then removed. The ear mold is then tested to make sure that it fits comfortably. If feedback is still present, the stent is removed, and a second stent of a larger size is installed. On the other hand, if the patient complains of discomfort, the stent can be removed and a smaller stent inserted.

The stent is removed by the stent removal or extraction tool, FIG. 8. The extraction tool stem is slowly guided into the bore and through the stent, FIG. 8a. The tool is then tilted, FIG. 8b, so that the hook end of the slot engages the end of the stent. The stent is then removed, FIG. 8c, by withdrawing the tool from the tubular passage.

Thus, there has been provided, a stent for use in an ear mold system and tools for inserting and removing the stent in an ear mold tubular passage. The stent permits adjustment of the outer diameter of an ear mold so that a mold can be snuggly fitted into the ear canal and prevent feedback.

What is claimed is:

1. A stented ear mold comprising an ear mold adapted to be fitted into the ear canal of a user, said ear mold including a tubular passage for conduction of sound from a hearing aid to the ear canal and ear drum; and
a stent inserted into said passage to cause the ear mold diameter to increase so that the mold can fit into the ear canal securely and prevent sound feedback.

2. A stented ear mold as in claim 1 in which said stent includes inclined ends for easy insertion and removal of the stent from the passage and maintains the tubular passage with seamless transitions.

3. A stented ear mold as in claim 2 in which said stent includes barbs for securing the stent along the ear mold passage.

4. A stent for insertion into an ear mold passage to adjust the our diameter of the ear mold comprising a tubular member having a central passage, said member having inclined ends.

5. A stent as in claim 4 which includes barbs.
